(19) United States
(12) Patent Application Publication
(10) Pub. No.: US 2023/0088684 A1
(43) Pub. Date: Mar. 23, 2023

(54) Processes for Producing Alkyl Salicylic Acids and Overbased Detergents Derived Therefrom

(71) Applicant: The Lubrizol Corporation, Wickliffe, OH (US)

(72) Inventors: Edward P. Sampler, Leicestershire (GB); John-Louis DiFlavio, Mentor, OH (US); Gary M. Walker, Allestree (GB); James P. Roski, Mentor, OH (US); Hyungsoo Kim, Hudson, OH (US)

(21) Appl. No.: 17/796,765

(22) PCT Filed: Jan. 29, 2021
(86) PCT No.: PCT/US21/15606

§ 371 Date: Aug. 1, 2022

Related U.S. Application Data
(60) Provisional application No. 62/968,557, filed on Jan. 31, 2020.

(51) Int. Cl. C07C 51/41; C07C 51/47; C07C 51/48; C10M 129/54; C10M 159/22; C10M 177/00

(52) U.S. Cl. CPC C07C 51/412; C07C 51/47; C07C 51/48; C10M 129/54; C10M 159/22; C10M 177/00; C10N 2030/04

(57) ABSTRACT

Alkyl salicylic acids, overbased detergents derived from alkyl salicylic acids, lubricating compositions including the alkyl salicylic acids and/or overbased detergents, and processes of making and using the same, are described.

### TECHNICAL FIELD

The disclosed technology relates to alkyl salicylic acids, overbased detergents derived from alkyl salicylic acids, lubricating compositions including the alkyl salicylic acids and/or the overbased detergents, and processes of making and using the same.

### BACKGROUND

Alkyl salicylic acids may be used to produce overbased detergents for use in, for example, lubricating compositions. It has been found that alkyl salicylic acids are thermally sensitive and may undergo thermal decarboxylation at temperatures greater than 150° C. to generate alkylphenates, which may be undesirable and/or have an undesired effect on resulting detergents. The disclosed technology provides, in part, control of temperature during the overbasing process to produce overbased detergents with superior performance properties as compared to conventional overbased detergents.

### SUMMARY

The subject matter disclosed herein provides a process comprising: (a) contacting an alkyl phenol with an alkali metal base compound and carbon dioxide at a temperature of from 120° C. to 180° C. to form an alkyl salicylic acid salt of the alkali metal base compound; (b) contacting the result of (a) with a mineral acid, in the absence of an additional non-polar hydrocarbyl liquid having a kinematic viscosity of less than 2.5 m²/s at 100° C., wherein the mineral acid and the alkali metal base compound form an alkali metal salt of the mineral acid, and wherein a solvent for the mineral acid is present in an amount sufficient to at least partially solubilize the alkali metal salt of the mineral acid to produce an alkyl salicylic acid and/or a salt thereof; and (c) removing at least 50 wt. % of the solvent and the alkali metal salt of the mineral acid from the result of (b), based on the total amount of solvent and alkali metal salt present in the result of (b). The process may further comprise (d) contacting the result of (c) with: (i) an alkaline earth metal oxide/hydroxide and/or a group II metal oxide/hydroxide, or a reactive equivalent thereof; (ii) carbon dioxide; and (iii) an alcohol, polyol, and/or (poly)ether; to produce an overbased detergent having a total base number (“TBN”) of at least 130 mg KOH/g on an active basis.

Also provided are: alkyl salicylic acids and/or overbased detergents prepared according to the above-described processes; compositions including the alkyl salicylic acids, derivatives thereof, and/or the overbased detergents; and/or lubricating compositions comprising the alkyl salicylic acids, derivatives thereof, and/or the overbased detergents.

The following embodiments of the present subject matter are contemplated:

1. A process comprising: (a) contacting an alkyl phenol with an alkali metal base compound and carbon dioxide at a temperature of from 120° C. to 180° C. to form an alkyl salicylic acid salt of the alkali metal base compound; (b) contacting the result of (a) with a mineral acid, in the absence of an additional non-polar hydrocarbyl liquid having a kinematic viscosity of less than 2.5 m²/s at 100° C., wherein the mineral acid and the alkali metal base compound form an alkali metal salt of the mineral acid, and wherein a solvent for the mineral acid is present in an amount sufficient to at least partially solubilize the alkali metal salt of the mineral acid to produce an alkyl salicylic acid and/or a salt thereof; and (c) removing at least 50 wt. % of the solvent and the alkali metal salt of the mineral acid from the result of (b), based on the total amount of solvent and alkali metal salt present in the result of (b).

2. The process of embodiment 1, wherein said removing the solvent comprises: (i) heating the result of (b) to a temperature sufficient to at least partially vaporize the solvent in the result of (b) and to form a precipitate of the alkali metal salt of the mineral acid, followed by removal of the precipitate, optionally wherein said removal is via filtration; and/or (ii) liquid-liquid phase separation.

3. The process of embodiment 2, wherein said heating is performed at a temperature of from 60° C. to 160° C., optionally under vacuum.

4. The process of any one of embodiments 1 to 3, wherein the alkali metal base compound is present in an amount of from 0.5 to 1.5 reactive equivalents based on the amount of the alkyl phenol.

5. The process of any one of embodiments 1 to 4, wherein the mineral acid is present in an amount of at least 0.9 equivalents of strong acid, based on the amount of alkali metal base compound.

6. The process of any one of embodiments 1 to 5, wherein a diluent oil is present in step (a) in an amount of from 10 wt. % to 50 wt. %, based on the total weight of all components present in step (a).

7. The process of any one of embodiments 1 to 6, wherein the mineral acid is a polyprotic mineral acid.

8. The process of any one of embodiments 1 to 6, wherein the mineral acid comprises at least one of sulfuric acid, hydrochloric acid, or phosphoric acid.

9. The process of any one of embodiments 1 to 8, wherein the solvent is present in an amount of from 0.1 wt. % to 70 wt. %, based on the total weight of all components present in step (b).

10. The process of any one of embodiments 1 to 9, wherein the alkyl phenol comprises at least one of 4-substituted alkyl phenol, 2-substituted alkyl phenol, or 2,4-disubstituted alkyl phenol.

11. The process of any one of embodiments 1 to 10, wherein the alkali metal base comprises at least one of sodium hydroxide, potassium hydroxide, or lithium hydroxide.

12. The process of any one of embodiments 1 to 11, further comprising: (d) contacting the result of (c) with: (i) an alkaline earth metal oxide/hydroxide and/or a group II metal oxide/hydroxide, or a reactive equivalent thereof; (ii) carbon dioxide; and (iii) an alcohol, polyol, and/or (poly)ether; to produce an overbased detergent having a total base number of at least 130 mg KOH/g on an active basis.

13. The process of embodiment 12, wherein the alkaline earth metal oxide/hydroxide and/or group II metal oxide/hydroxide, or a reactive equivalent thereof, is added in an amount of 1.1 to 8 reactive equivalents based on the alkyl salicylic acid.

14. The process of either embodiment 12 or embodiment 13, wherein the alkaline earth metal oxide/hydroxide comprises at least one of calcium oxide, calcium hydroxide, magnesium oxide, or magnesium hydroxide.

15. The process of any one of embodiments 12 to 14, wherein the group II metal oxide/hydroxide comprises zinc oxide.

16. The process of any one of embodiments 12 to 15, wherein the alcohol, polyol, and/or (poly)ether is present in an amount of from 5 wt. % to 30 wt. %, based on the total weight of all components present in step (d).

17. The process of any one of embodiments 12 to 16, wherein the alcohol, polyol, and/or (poly)ether comprises at least one of ethylene glycol or propylene glycol.

18. The process of any one of embodiments 12 to 17, wherein the total base number of the overbased detergent is from 130 mg KOH/g to 600 mg KOH/g on an active basis.

19. The process of any one of embodiments 12 to 18, wherein the overbased detergent has a metal ratio of from 1.5 to 10.

20. An alkyl salicylic acid prepared according to any one of embodiments 1 to 11.

21. A composition comprising at least one derivative of the alkyl salicylic acid of embodiment 20.

22. An overbased detergent prepared according to any one of embodiments 12 to 19.

23. A lubricating composition comprising at least one of: (i) the alkyl salicylic acid of embodiment 20; (ii) the composition of embodiment 21; or the overbased detergent of embodiment 22.

24. The lubricating composition of embodiment 23, further comprising an oil of lubricating viscosity in an amount of 30 wt. % to 95 wt. %, based on the total weight of the composition, and optionally at least one of an ashless succinimide dispersant, a phosphorous-containing anti-wear agent, an ashless antioxidant, or a polymeric viscosity modifier.

25. The lubricating composition of embodiment 24, further comprising an ashless succinimide dispersant in an amount of from 0.5 wt. % to 7 wt. %, based on the total weight of the composition.

26. The lubricating composition of either embodiment 24 or embodiment 25, further comprising a phosphorous-containing anti-wear agent in an amount of from 0.1 wt. % to 2 wt. %, based on the total weight of the composition.

27. The lubricating composition of any one of embodiments 24-26, further comprising an ashless antioxidant in an amount of from 0.1 wt. % to 5 wt. %, based on the total weight of the composition.

28. The lubricating composition of any one of embodiments 24-27, further comprising a polymeric viscosity modifier in an amount of from 0.1 wt. % to 3 wt. %, based on the total weight of the composition.

### DETAILED DESCRIPTION

Various features and embodiments of the present subject matter will be described below by way of non-limiting illustration.

The amount of each chemical component described herein is presented exclusive of any solvent or diluent oil, which may be customarily present in the commercial material, that is, on an active chemical basis, unless otherwise indicated. However, unless otherwise indicated, each chemical or composition referred to herein should be interpreted as being a commercial grade material which may contain the isomers, by-products, derivatives, and other such materials which are normally understood to be present in the commercial grade.

As used herein, the term “hydrocarbyl substituent” or “hydrocarbyl group” (or in appropriate context, simply “hydrocarbyl”) is used in its ordinary sense, which is well-known to those skilled in the art. Specifically, it refers to a group having a carbon atom directly attached to the remainder of the molecule and having predominantly hydrocarbon character. Examples of hydrocarbyl groups include:

hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form a ring);

substituted hydrocarbon substituents, that is, substituents containing non-hydrocarbon groups which, in the context of the present subject matter, do not alter the predominantly hydrocarbon nature of the substituent (e.g., halo (especially chloro and fluoro), hydroxy, alkoxy, mercapto, alkylmercapto, nitro, nitroso, and sulfoxy);

hetero substituents, that is, substituents which, while having a predominantly hydrocarbon character, in the context of the present subject matter, contain other than carbon in a ring or chain otherwise composed of carbon atoms and encompass substituents as pyridyl, furyl, thienyl and imidazolyl. Heteroatoms include sulfur, oxygen, and nitrogen. In general, no more than two, or no more than one, non-hydrocarbon substituent will be present for every ten carbon atoms in the hydrocarbyl group; alternatively, there may be no non-hydrocarbon substituents in the hydrocarbyl group.

It is known that some of the materials described herein may interact in the final formulation, so that the components of the final formulation may be different from those that are initially added. For instance, metal ions (of, e.g., a detergent) may migrate to other acidic or anionic sites of other molecules. The products formed thereby, including the products formed upon employing the composition of the present subject matter in its intended use, may not be susceptible of easy description. Nevertheless, all such modifications and reaction products are included within the scope of the present subject matter; the present subject matter encompasses the composition prepared by admixing the components described herein.

As used herein, the indefinite article “a” is intended to mean one or more than one. As used herein, the phrase “at least one” means one or more than one of the following terms. Thus, “a” and “at least one” may be used interchangeably. For example “at least one of A, B or C” means that just one of A, B or C may be included, and any mixture of two or more of A, B and C may be included, in alternative embodiments.

As used herein, the term “about” means that a value of a given quantity is within ±20% of the stated value. In other embodiments, the value is within ±15% of the stated value. In other embodiments, the value is within ±10% of the stated value. In other embodiments, the value is within ±5% of the stated value. In other embodiments, the value is within ±2.5% of the stated value. In other embodiments, the value is within ±1% of the stated value. In other embodiments, the value is within a range of the explicitly-described value which would be understood by those of ordinary skill, based on the disclosures provided herein, to perform substantially similarly to compositions including the literal amounts described herein.

As used herein, the term “substantially” means that a value of a given quantity is within ±10% of the stated value. In other embodiments, the value is within ±5% of the stated value. In other embodiments, the value is within ±2.5% of the stated value. In other embodiments, the value is within ±1% of the stated value.

As used herein, the term “substantially free of” means that a component does not include any intentional addition of the material which the component is “substantially free of”. For example, the component may include a material which the component is “substantially free of” at no more than impurity levels, which may be the result of incomplete chemical reactions and/or unintended/undesired (but perhaps unavoidable) reaction products.

As used herein, the transitional term “comprising,” which is synonymous with “including,” “containing,” or “characterized by,” is inclusive or open-ended and does not exclude additional, un-recited elements or method/process steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the essential or basic and novel characteristics of the composition or method under consideration.

Unless context dictates otherwise, use herein of parentheses around a term indicates that the term enclosed in the parentheses is optional. For example, the term “(poly)ether” means an ether and/or a polyether.

Provided is a process comprising: (a) contacting an alkyl phenol with an alkali metal base compound and carbon dioxide at a temperature of from 120° C. to 180° C. to form an alkyl salicylic acid salt of the alkali metal base compound; (b) contacting the result of (a) with a mineral acid, in the absence of an additional non-polar hydrocarbyl liquid having a kinematic viscosity of less than 2.5 m²/s at 100° C., wherein the mineral acid and the alkali metal base compound form an alkali metal salt of the mineral acid, and wherein a solvent for the mineral acid is present in an amount sufficient to at least partially solubilize the alkali metal salt of the mineral acid to produce an alkyl salicylic acid and/or a salt thereof; and (c) removing at least 50 wt. % of the solvent and the alkali metal salt of the mineral acid from the result of (b), based on the total amount of solvent and alkali metal salt present in the result of (b). The process may further comprise (d) contacting the result of (c) with: (i) an alkaline earth metal oxide/hydroxide and/or a group II metal oxide/hydroxide, or a reactive equivalent thereof; (ii) carbon dioxide; and (iii) an alcohol, polyol, and/or (poly)ether; to produce an overbased detergent having a total base number of at least 130 mg KOH/g on an active basis.

In certain embodiments, the process may consist essentially of: (a) contacting an alkyl phenol with an alkali metal base compound and carbon dioxide at a temperature of from 120° C. to 180° C. to form an alkyl salicylic acid salt of the alkali metal base compound; (b) contacting the result of (a) with a mineral acid, in the absence of an additional non-polar hydrocarbyl liquid having a kinematic viscosity of less than 2.5 m²/s at 100° C., wherein the mineral acid and the alkali metal base compound form an alkali metal salt of the mineral acid, and wherein a solvent for the mineral acid is present in an amount sufficient to at least partially solubilize the alkali metal salt of the mineral acid to produce an alkyl salicylic acid and/or a salt thereof; and (c) removing at least 50 wt. % of the solvent and the alkali metal salt of the mineral acid from the result of (b), based on the total amount of solvent and alkali metal salt present in the result of (b).

In certain embodiments, the process may consist of: (a) contacting an alkyl phenol with an alkali metal base compound and carbon dioxide at a temperature of from 120° C. to 180° C. to form an alkyl salicylic acid salt of the alkali metal base compound; (b) contacting the result of (a) with a mineral acid, in the absence of an additional non-polar hydrocarbyl liquid having a kinematic viscosity of less than 2.5 m²/s at 100° C., wherein the mineral acid and the alkali metal base compound form an alkali metal salt of the mineral acid, and wherein a solvent for the mineral acid is present in an amount sufficient to at least partially solubilize the alkali metal salt of the mineral acid to produce an alkyl salicylic acid and/or a salt thereof; and (c) removing at least 50 wt. % of the solvent and the alkali metal salt of the mineral acid from the result of (b), based on the total amount of solvent and alkali metal salt present in the result of (b).

In certain embodiments, the process may consist essentially of: (a) contacting an alkyl phenol with an alkali metal base compound and carbon dioxide at a temperature of from 120° C. to 180° C. to form an alkyl salicylic acid salt of the alkali metal base compound; (b) contacting the result of (a) with a mineral acid, in the absence of an additional non-polar hydrocarbyl liquid having a kinematic viscosity of less than 2.5 m²/s at 100° C., wherein the mineral acid and the alkali metal base compound form an alkali metal salt of the mineral acid, and wherein a solvent for the mineral acid is present in an amount sufficient to at least partially solubilize the alkali metal salt of the mineral acid to produce an alkyl salicylic acid and/or a salt thereof; (c) removing at least 50 wt. % of the solvent and the alkali metal salt of the mineral acid from the result of (b), based on the total amount of solvent and alkali metal salt present in the result of (b); and (d) contacting the result of (c) with: (i) an alkaline earth metal oxide/hydroxide and/or a group II metal oxide/hydroxide, or a reactive equivalent thereof; (ii) carbon dioxide; and (iii) an alcohol, polyol, and/or (poly)ether; to produce an overbased detergent having a total base number of at least 130 mg KOH/g on an active basis.

In certain embodiments, the process may consist of: (a) contacting an alkyl phenol with an alkali metal base compound and carbon dioxide at a temperature of from 120° C. to 180° C. to form an alkyl salicylic acid salt of the alkali metal base compound; (b) contacting the result of (a) with a mineral acid, in the absence of an additional non-polar hydrocarbyl liquid having a kinematic viscosity of less than 2.5 m²/s at 100° C., wherein the mineral acid and the alkali metal base compound form an alkali metal salt of the mineral acid, and wherein a solvent for the mineral acid is present in an amount sufficient to at least partially solubilize the alkali metal salt of the mineral acid to produce an alkyl salicylic acid and/or a salt thereof; (c) removing at least 50 wt. % of the solvent and the alkali metal salt of the mineral acid from the result of (b), based on the total amount of solvent and alkali metal salt present in the result of (b); and (d) contacting the result of (c) with: (i) an alkaline earth metal oxide/hydroxide and/or a group II metal oxide/hydroxide, or a reactive equivalent thereof; (ii) carbon dioxide; and (iii) an alcohol, polyol, and/or (poly)ether; to produce an overbased detergent having a total base number of at least 130 mg KOH/g on an active basis.

As used herein, the term “alkyl salicylic acid” means a salicylic acid including at least one alkyl group pendant from the ring portion of the salicylic acid. For example, the alkyl salicylic acid may be of the general formula:

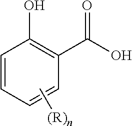

wherein n is 1 to 3; and R is a branched or linear hydrocarbyl group of 14 to 32 carbon atoms, or 16 to 24 carbon atoms, or 18 to 22 carbon atoms.

In certain embodiments, contacting the alkyl phenol with carbon dioxide includes providing enough carbon dioxide such that at least 50 mol %, at least 55 mol %, at least 60 mol %, at least 65 mol %, at least 70 mol %, or at least 75 mol %, of the alkyl phenol is converted into alkyl salicylic acid.

As used herein, the term “mineral acid” refers to any inorganic acid.

As used herein, the term “non-polar hydrocarbyl liquid” may mean, or in the alternative may include, non-water-miscible hydrocarbyl liquid.

In certain embodiments, step (c) may comprise removing all or substantially all of the solvent and the alkali metal salt of the mineral acid from the result of (b), based on the total amount of solvent and alkali metal salt present in the result of (b). In certain embodiments, removing substantially all of the alkali metal salt means that no more than 500 ppm by weight of the alkali metal salt will be present in the result of (c).

The phrase “a solvent for the mineral acid is present in an amount sufficient to at least partially solubilize the alkali metal salt of the mineral acid” is intended to mean that at least a substantial portion of the alkali metal salt of the mineral acid is solubilized in the solvent for the mineral acid. In certain embodiments, at least 15 (such as 20, 25, 30, 35, 40, 45, 50, 55, 60, 65, 70, 75, 80, 85, 90, 95, 96, 97, 98, 99, or 100) wt. % of the alkali metal salt of the mineral acid is solubilized in the solvent for the mineral acid. In certain embodiments, substantially all of the alkali metal salt of the mineral acid is solubilized in the solvent for the mineral acid.

In certain embodiments, said removing the solvent comprises: (i) heating the result of (b) to a temperature sufficient to at least partially vaporize the solvent in the result of (b) and to form a precipitate of the alkali metal salt of the mineral acid, followed by removal of the precipitate, optionally wherein said removal is via filtration; and/or (ii) liquid-liquid phase separation. A suitable non-limiting method for liquid-liquid phase separation includes extraction of the salt(s) from the oleaginous phase (such as the oil phase) to the aqueous phase (such as the water phase), followed by mechanical separation or gravitational separation of the phases. In certain embodiments, said heating is performed at a temperature of from 60° C. to 160° C., such as from 80° C. to 120° C., optionally under vacuum. In certain embodiments in which said heating is performed at lower temperatures, said heating must be performed under vacuum. For example, if the temperature is lower than the vaporization temperature of the particular solvent at ambient conditions, operating under vacuum may be required to at least partially vaporize the solvent. In certain embodiments, said liquid-liquid phase separation may be carried out at temperatures of from 60° C. to 90° C., such as from 80° C. to 90° C. As used in this paragraph, “at least partially vaporize” may mean that a substantial portion of the solvent is vaporized, such as at least 15% (such as 20%, 25%, 30%, 35%, 40%, 45%, 50%, 55%, 60%, 65%, 70%, 75%, 80%, 85%, 90%, 95%, 96%, 97%, 98%, 99% or 100%) based on the total amount of solvent present prior to removal of the solvent. In certain embodiments, removing the solvent comprises heating the result of (b) to a temperature sufficient to vaporize substantially all of the solvent in the result of (b).

In certain embodiments, the alkali metal base compound is present in an amount of from 0.5 (such as 0.55, 0.6, 0.65, 0.7, 0.75, 0.8, 0.85, 0.9, or 0.95) to 1.5 (such as 1.45, 1.4, 1.35, 1.3, 1.25, 1.2, 1.15, 1.1, or 1.05) reactive equivalents based on the amount of the alkyl phenol. In certain embodiments, the alkali metal base compound is present in an amount of 1 reactive equivalent based on the amount of alkyl phenol.

In certain embodiments, the mineral acid is present in an amount of at least 0.9 equivalents (such as 0.91, 0.92, 0.93, 0.94, 0.95, 0.96, 0.97, 0.98, 0.99, 1, 1.01, or 1.02 equivalents) of strong acid, based on the amount of alkali metal base compound. In certain embodiments, the mineral acid is present in an amount of from 0.9 equivalents (such as 0.91, 0.92, 0.93, 0.94, 0.95, 0.96, 0.97, 0.98, 0.99, 1, 1.01, or 1.02 equivalents) to 3 equivalents (such as 2.9, 2.8, 2.7, 2.6, 2.5, 2.4, 2.3, 2.2, 2.1, 2, 1.9, 1.8, 1.7, 1.6, 1.5, 1.45, 1.4, 1.35, 1.3, 1.25, 1.2, 1.19, 1.18, 1.17, 1.16, 1.15, 1.14, 1.13, 1.12, 1.11, 1.1, 1.09, 1.08, 1.07, 1.06, 1.05, or 1.04 equivalents) of strong acid, based on the amount of alkali metal base compound. As used in these embodiments, the term “equivalent of strong acid” means each proton of the acid having a pKₐ of less than 3.

In certain embodiments, a diluent oil is present in step (a) in an amount of from 10 wt. % to 50 wt. %, based on the total weight of all components present in step (a). In certain embodiments, the diluent oil has a kinematic viscosity of greater than 2.5 m²/s at 100° C.

In certain embodiments, the mineral acid is a polyprotic mineral acid. In certain embodiments, the mineral acid comprises at least one of sulfuric acid, hydrochloric acid, or phosphoric acid.

In certain embodiments, the solvent is present in an amount of from 0.1 wt. % to 70 wt. %, based on the total weight of all components present in step (b). In certain embodiments, the solvent is present in an amount of from 0.1 (such as 0.2, 0.3, 0.4, 0.5, 0.6, 0.7, 0.8, 0.9, 1, 1.5, 2, 2.5, 3, 3.5, 4, 4.5, 5, 6, 7, 8, 9, or 10) wt. % to 70 (such as 65, 60, 55, 50, 45, 40, 35, 30, 25, 24, 23, 22, 21, 20, 19, 18, 17, 16, 15, 14.5, 14, 13.5, 13, 12.5, or 12) wt. %, based on the total weight of all component present in step (b)

In certain embodiments, the mineral acid may be provided as a solution including the solvent for the mineral acid described above. In certain embodiments, the solvent may be water and/or a water-miscible solvent. In certain embodiments, the solvent may comprise at least one of water, methanol, ethanol, isopropanol, ethylene glycol, or propylene glycol. In certain embodiments, the solvent comprises water. In conventional methods of producing alkyl salicylic acids, non-polar hydrocarbyl solvents, such as xylene, have been used. As discussed herein, the present subject matter provides benefits derived at least in part from eliminating use of such non-polar hydrocarbyl solvents.

In certain embodiments, the alkyl phenol comprises at least one of 4-substituted alkyl phenol, 2-substituted alkyl phenol, or 2,4-disubstituted alkyl phenol. In certain embodiments, an alkyl group of each alkyl phenol molecule may independently be a C₁₄-C₃₂ (such as C₁₆-C₂₄) linear or branched alkyl group. In embodiments in which the alkyl phenol includes at least two alkyl groups, each alkyl phenol molecule may also include a C₁-C₃₂ linear or branched alkyl group. In certain embodiment, the alkyl phenol may comprise at least one 4-substituted alkylphenol comprising at least one of 4-tetradecyl phenol, 4-hexadecyl phenol, 4-cetylphenol, 4-octadecyl phenol, 4-sebacyl phenol, 4-eicosylphenol, or 4-docosylphenol. In certain embodiments, the alkylphenol may comprise a 2-alkylphenol, wherein the alkyl group is as above with regard to the 4-alkyphenols (e.g., C₁₄, C₁₆, C₁₈, C₂₀, C₂₂, or mixtures thereof). In certain embodiments, 2,4-disubstituted phenols are selected such that at least one of the 2- or 4-alkyl group (the primary alkyl group) comprises at least one of tetradecyl-, hexadecyl-, octadecyl-, eicosyl-, or docosyl-groups; the second alkyl group may be the same or different as the primary alkyl group and/or may be at least one of methyl, propyl, or butyl.

In certain embodiments, the alkali metal base comprises at least one of sodium hydroxide, potassium hydroxide, or lithium hydroxide.

As used herein, “reactive equivalents” of the alkaline earth metal oxide/hydroxide and/or a group II metal oxide/hydroxide may include any such known reactive equivalents. For example, such reactive equivalents may include other metal salts of weak acids, such as carbonates or carbamates.

In certain embodiments, the alkaline earth metal oxide/hydroxide and/or group II metal oxide/hydroxide, or reactive equivalent thereof, and carbon dioxide are present in an amount sufficient to produce a metal ratio of at least 1.5 in the resulting overbased detergent. In certain embodiments, the overbased detergent has a metal ratio of 1.5 to 10, 1.8 to 10, 2.5 to 8, or 3.5 to 5.

As used herein, the term “overbased” detergent means a detergent which contains a stoichiometric excess of a metal base for the acidic organic substrate. This principle is related to the term “metal ratio”, which is the ratio of the total equivalents of the metal to the equivalents of the acidic organic compound. A neutral metal salt has a metal ratio of 1.3 or less, or about 1. A salt having 4.5 times as much metal as present in a normal salt will have metal excess of 3.5 equivalents, or a ratio of 4.5. The term “metal ratio” is also explained in “Chemistry and Technology of Lubricants”, Third Edition, Edited by R. M. Mortier and S. T. Orszulik, 2010, page 219, sub-heading 7.25.

As used herein, the term “active basis”, when referring to the total base number (“TBN”) of the overbased detergent, means the TBN of the overbased detergent complex itself corrected for any diluent, such as mineral or other oils.

In certain embodiments, the alkaline earth metal oxide/hydroxide and/or group II metal oxide/hydroxide, or a reactive equivalent thereof, is added in an amount of 1.1 (such as 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3) to 8 (such as 7.5, 7, 6.5, 6, 5.5, 5, 4.9, 4.8, 4.7, 4.6, 4.5, 4.4, 4.3, 4.2, 4.1, or 4) reactive equivalents based on the alkyl salicylic acid.

In certain embodiments, the alkaline earth metal oxide/hydroxide comprises at least one of calcium oxide, calcium hydroxide, magnesium oxide, or magnesium hydroxide.

In certain embodiments, the group II metal oxide/hydroxide comprises zinc oxide.

In certain embodiments, the alcohol, polyol, and/or (poly)ether is present in an amount of from 5 (such as 6, 7, 8, 9, 10, 11, or 12) wt. % to 30 (such as 25, 20, 19, 18, 17, 16, 15, 14, 13, or 12) wt. %, based on the total weight of all components present in step (d).

In certain embodiments, the alcohol, polyol, and/or (poly)ether comprises at least one of methanol, ethanol, n-propanol, isopropanol, t-butanol, glycerol, tetrahydrofuran, ethylene glycol, or propylene glycol.

In certain embodiments, the total base number (measured by ASTM D2896) of the overbased detergent is from 130 (such as 140, 150, 160, 170, 180, 190, 200, 210, 220, 230, 240, 250, 260, 270, 280, 290, or 300) mg KOH/g to 600 (such as 590, 580, 570, 560, 550, 540, 530, 520, 510, 500, 490, 480, 470, 460, 450, 440, 430, 420, 410, 400, 390, 380, 370, 360, or 350) mg KOH/g on an active basis. When referring to total base number herein, it has been measured using ASTM D2896, unless indicated otherwise.

The processes described herein may be conducted via batch, semi-continuous, and/or continuous processes.

Provided are alkyl salicylic acids prepared according to the processes described above. Also provided are compositions comprising at least one derivative of the alkyl salicylic acids prepared according to the processes described above. As used in the previous sentence “derivatives” of the alkyl salicylic acids may include, but are not limited to, at least one of metal salts, amine salts, ammonium salts (such as quaternary ammonium salts), overbased amine salts, or borate ester complexes of the alkyl salicylic acids.

Also provided are overbased detergents prepared according to the processes described above.

The processes described above, and the compositions made therefrom, may be used in lubricating compositions. Such lubricating compositions may comprise an oil of lubricating viscosity in an amount of 30 wt. % to 95 wt. %, based on the total weight of the composition, and optionally at least one of an ashless succinimide dispersant, a phosphorous-containing anti-wear agent, an ashless antioxidant, or a polymeric viscosity modifier.

### Oils of Lubricating Viscosity

The oils of lubricating viscosity may include, for example, natural and synthetic oils, oils derived from hydrocracking, hydrogenation, and hydrofinishing, unrefined, refined and re-refined oils, and mixtures thereof. Oils of lubricating viscosity may also be defined as specified in the American Petroleum Institute (API) Base Oil Interchangeability Guidelines.

Unrefined oils are those obtained directly from a natural or synthetic source generally without (or with little) further purification treatment. Refined oils are similar to the unrefined oils except they have been further treated in one or more purification steps to improve one or more properties. Purification techniques are known in the art and include solvent extraction, secondary distillation, acid or base extraction, filtration, percolation and the like. Re-refined oils are also known as reclaimed or reprocessed oils and are obtained by processes similar to those used to obtain refined oils and often are additionally processed by techniques directed to removal of spent additives and oil breakdown products. Natural oils useful in making the inventive lubricants include animal oils, vegetable oils (e.g., castor oil), mineral lubricating oils (such as liquid petroleum oils and solvent-treated or acid-treated mineral lubricating oils of the paraffinic, naphthenic or mixed paraffinic-naphthenic types), and oils derived from coal or shale or mixtures thereof. Synthetic lubricating oils are useful and include: hydrocarbon oils such as polymerised and interpolymerised olefins (e.g., polybutylenes, poly-propylenes, propyleneisobutylene copolymers); poly(1-hexenes), poly(1-octenes), poly(1-decenes), and mixtures thereof; alkyl-benzenes (e.g., dodecylbenzenes, tetradecylbenzenes, dinonylbenzenes, di-(2-ethylhexyl)-benzenes); polyphenyls (e.g., biphenyls, terphenyls, alkylated polyphenyls); diphenyl alkanes, alkylated diphenyl alkanes, alkylated diphenyl ethers and alkylated diphenyl sulphides and their derivatives; analogs and homologs thereof; or mixtures thereof. Other synthetic lubricating oils include polyol esters (such as Priolube® 3970), diesters, liquid esters of phosphorus-containing acids (e.g., tricresyl phosphate, trioctyl phosphate, and the diethyl ester of decane phosphonic acid), or polymeric tetrahydrofurans. Synthetic oils may be produced by Fischer-Tropsch reactions and typically may be hydroisomerised Fischer-Tropsch hydrocarbons or waxes. In one aspect, oils may be prepared by a Fischer-Tropsch gas-to-liquid synthetic procedure as well as other gas-to-liquid processes.

Oils of lubricating viscosity may also be defined as specified in the American Petroleum Institute (API) Base Oil Interchangeability Guidelines. The five base oil groups are as follows: Group I (sulfur content >0.03 wt. %, and/or <90 wt. % saturates, viscosity index 80-120); Group II (sulphur content ≤0.03 wt. %, and >90 wt. % saturates, viscosity index 80-120); Group III (sulphur content ≤0.03 wt. %, and >0.90 wt. % saturates, viscosity index ≥120); Group IV (all polyalphaolefins); and Group V (all others not included in Groups I, II, III, or IV). The oil of lubricating viscosity comprises at least one of a Group I oil, a Group II oil, a Group III oil, a Group IV oil, or a Group V oil. In certain embodiments, just one of these oils, or a mixture of two or more of them, may be desirable for a particular end use of the composition including the oil, as is known by those of skill in the art.

### Dispersants

In certain embodiments, lubricating compositions described herein may comprise an ashless succinimide dispersant in an amount of from 0.5 wt. % to 7 wt. %, based on the total weight of the composition.

Dispersants are well-known in the field of lubricants and include primarily what are known as ashless dispersants and/or polymeric dispersants. Ashless dispersants are so-called because, as supplied, they do not contain metal and thus do not normally contribute to sulfated ash when added to a lubricant. However, they may, of course, interact with ambient metals once they are added to a lubricant which includes metal-containing species. Ashless dispersants are characterized by a polar group attached to a relatively high molecular weight hydrocarbon chain. Typical ashless dispersants include N-substituted long-chain alkenyl succinimides, having a variety of chemical structures including those conforming to formula (IX):

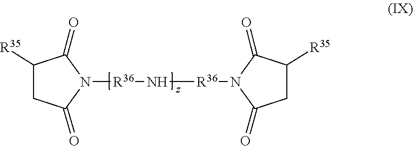

wherein in one aspect, each R³⁵ is independently an alkyl group, and in certain embodiments, a polyisobutylene group with a molecular weight (Mₙ) of 500-5000 based on the polyisobutylene precursor, and R³⁶ is an alkylene group, such as an ethylene group. Such molecules are commonly derived from reaction of an alkenyl acylating agent with a polyamine, and a wide variety of linkages between the two moieties is possible beside the simple imide structure shown above, including a variety of amides and quaternary ammonium salts. In the above structure, the amine portion is shown as an alkylene polyamine, although other aliphatic and aromatic monoamines and polyamines may also be used. Also, a variety of modes of linkage of the R³⁵ groups onto the imide structure are possible, including various cyclic linkages. The ratio of the carbonyl groups of the acylating agent to the nitrogen atoms of the amine may be 1:0.5 to 1:3, and in other instances 1:1 to 1:2.75, or 1:1.5 to 1:2.5. Succinimide dispersants are more fully described in U.S. Pat. Nos. 4,234,435 and 3,172,892, and in EP 0 355 895 B1.

Another class of ashless dispersant is high molecular weight esters. These materials are similar to the above-described succinimides except that they may be prepared by reaction of a hydrocarbyl acylating agent and a polyhydric aliphatic alcohol such as glycerol, pentaerythritol, or sorbitol. Such materials are described in more detail in U.S. Pat. No. 3,381,022.

Another class of ashless dispersant is Mannich bases. These are materials which are formed by the condensation of a higher molecular weight, alkyl substituted phenol, an alkylene polyamine, and an aldehyde such as formaldehyde. Such materials may have general structure (X):

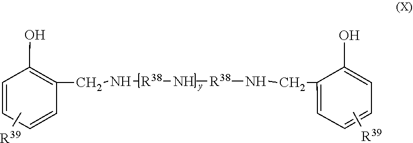

wherein R³⁸ is an alkylene group, e.g., an ethylene group; and R³⁹ is a hydrocarbyl substituent having from about 40 to about 20,000 carbon atoms, or from about 80 to about 250 carbon atoms. In one aspect, R³⁹ is selected from polyisobutyl and polypropyl substitutents derived from the alkylation of the phenol moiety with polybutylenes or polypropylenes. The foregoing Mannich base dispersants described in more detail in U.S. Pat. No. 3,634,515.

Other dispersants include polymeric dispersant additives, which are generally hydrocarbon-based polymers which contain polar functionality to impart dispersancy characteristics to the polymer.

Dispersants can also be post-treated by reaction with any of a variety of agents. Among these are urea, thiourea, dimercaptothiadiazoles, carbon disulfide, aldehydes, ketones, carboxylic acids, hydrocarbon-substituted succinic anhydrides, nitriles, epoxides, boron compounds, and phosphorus compounds. References detailing such treatment are disclosed in U.S. Pat. No. 4,654,403.

In one embodiment, the dispersant is a polyisobutenyl succinimide dispersant that has been post-treated with a boron compound, i.e. a borated dispersant. In one embodiment, the lubricating composition comprises at least one non-boron containing dispersant, or at least one boron-containing dispersant, or combinations thereof.

The amount of the dispersant in a fully formulated lubricant of the present technology may be at least 0.1% of the lubricant composition, or at least 0.3 wt. %, 0.5 wt. %, 1 wt. %, or 2 wt. %, and in certain embodiments, at most 9 wt. %, or 8 wt. %, or 6 wt. %, or 4 wt. %, or 3 wt. %, or 2 wt. %, based on the weight of the total composition.

The amount of borated dispersant may also be represented in the amount of boron from the dispersant present in the composition. In one embodiment, a borated dispersant may be present in an amount to deliver from 25 ppm boron to 1100 ppm boron, from 35 ppm boron to 500 ppm boron, from 50 ppm boron to 300 ppm boron, or from 100 ppm to 450 ppm boron to the lubricant composition.

### Other Detergents

Lubricating compositions in accordance with the present invention may contain one or more detergents in addition to the inventive detergent. Detergents used in lubricating compositions may be overbased materials, sometimes referred to as overbased or superbased salts, which are generally homogeneous Newtonian systems having a metal content in excess of that which would be present for neutralization according to the stoichiometry of the metal and the detergent anion. The amount of excess metal is commonly expressed in terms of metal ratio, that is, the ratio of the total equivalents of the metal to the equivalents of the acidic organic compound. Overbased materials are prepared by reacting an acidic material (such as carbon dioxide) with an acidic organic compound, an inert reaction medium (e.g., mineral oil), a stoichiometric excess of a metal base or a quaternary ammonium base, and a promoter such as a phenol or alcohol. The acidic organic material will normally have a sufficient number of carbon atoms to provide oil-solubility.

Overbased detergents can be characterized by their TBN, the amount of strong acid needed to neutralize all of the material's basicity, which may be expressed as mg KOH per gram of sample (“mg KOH/g”). Since overbased detergents are commonly provided in a form which contains diluent oil, for the purpose of this document, TBN is to be recalculated (when referring to a detergent or specific additive) to an oil-free basis. Some useful detergents may have a TBN of 100 to 800, 150 to 750, or 400 to 700 mg KOH/g.

The metal compounds useful in making the basic metal salts are generally any Group 1 or Group 2 metal compounds (based on the CAS version of the Periodic Table of the Elements). Examples include alkali metals such as sodium, potassium, lithium, copper, magnesium, calcium, barium, zinc, and cadmium. In certain embodiments, the metals are sodium, magnesium, or calcium. The anionic portion of the salt can be hydroxide, oxide, carbonate, borate, or nitrate.

In certain embodiments, the lubricant can contain an overbased sulfonate detergent. Suitable sulfonic acids include sulfonic and thiosulfonic acids, including mono or polynuclear aromatic or cyclo-aliphatic compounds. Certain oil-soluble sulfonates can be represented by R¹⁰-T(SO₃⁻)ₐ or R¹¹(SO₃⁻)_{b}, where a and b are each at least one; T is a cyclic nucleus such as benzene or toluene; R¹⁰ is an aliphatic group such as alkyl, alkenyl, alkoxy, or alkoxyalkyl; (R¹⁰)-T typically contains a total of at least 15 carbon atoms; and R¹¹ is an aliphatic hydrocarbyl group typically containing at least 15 carbon atoms. The groups T, R¹⁰, and R¹¹ can also contain other inorganic or organic substituents. In one aspect, the sulfonate detergent may be a predominantly linear alkylbenzene sulfonate detergent having a metal ratio of at least 6 or at least 8 as described in paragraphs [0022] to [0037] of US 2005/0065045 A1. In some aspects, the linear alkyl group may be attached to the benzene ring anywhere along the linear chain of the alkyl group, but often in the 2, 3 or 4 position of the linear chain, and in some instances predominantly in the 2 position.

Another overbased material is an overbased phenate detergent. The phenols useful in making phenate detergents can be represented by (R¹⁵)ₐ—Ar—(OH)_{b}, wherein R¹⁵ is an aliphatic hydrocarbyl group of 4 to 400, or 6 to 80, or 6 to 30, or 8 to 25, or 8 to 15 carbon atoms; Ar is an aromatic group such as benzene, toluene or naphthalene; a and b are each at least one, the sum of a and b being up to the number of displaceable hydrogens on the aromatic nucleus of Ar, such as 1 to 4 or 1 to 2. There is typically an average of at least 8 aliphatic carbon atoms provided by the R¹⁵ groups for each phenol compound. Phenate detergents are also sometimes provided as sulfur-bridged species. In one embodiment, the lubricant composition is free of or substantially free of (i.e., contains less than 0.05 weight percent) of a phenate detergent derived from a C₁₀ to C₁₄ alkylphenol.

In one embodiment of the present invention, a lubricating composition is provided which contains a blend of detergents comprising (A) a first detergent comprising an alkaline earth metal salt of a salicylic acid, an overbased metal salt of a salicylic acid, or combinations thereof and (B) a second detergent different from the first detergent. In one embodiment, the second detergent comprises a neutral or overbased metal salt of alkylbenzenesulfonic acid. In one embodiment, the first detergent is 25% to 75% by weight, for example 50% to 75% by weight, of the total detergent blend.

The amount of the total detergent, in the formulations of the present technology, is typically at least 0.6 weight percent on an oil-free basis, such as 0.7 to 5 weight percent, or 1 to 3 weight percent.

If the detergents in the lubricant composition are overbased, the amount of overbased detergent can also be represented by the amount of metal, specifically alkaline earth metal, delivered to the lubricating composition by the detergent. In one aspect, the overbased detergent is present in an amount to deliver 500 ppm to 3000 ppm, or 800 to 2400 ppm by weight alkaline earth metal to the composition, or combinations of alkaline earth metals. The overbased detergent may be present in an amount to deliver 1000 ppm to 2500 ppm calcium to the composition, or in an amount to deliver 400 ppm to 2500 ppm magnesium to the composition, or combinations thereof. In one embodiment, the lubricating composition comprises at least 400 ppm magnesium or at least 750 ppm magnesium, and no more than 1500 ppm calcium from overbased detergents.

### Anti-Wear Agents

The lubricant compositions of the disclosed technology can also contain anti-wear agent. Suitable anti-wear agents include metal-containing and metal-free phosphorus compounds, organic phosphorus-free and sulfur-free compounds, molybdenum compounds, phosphorus-free sulfur compounds, sulfur-free phosphorus compounds, and mixtures and combinations thereof.

In certain embodiments, such lubricating compositions may comprise a phosphorous-containing anti-wear agent in an amount of from 0.1 wt. % to 2 wt. %, based on the total weight of the composition.

In one aspect, the phosphorus-containing compound may be a metal salt of a phosphorus acid of the formula (XII):

[(R⁴³O)(R⁴⁴O)P(═S)(—S)]ₙ-M (XII)

wherein R⁴³ and R⁴⁴ are, independently, hydrocarbyl groups containing 3 to 30 carbon atoms, and can be obtained by heating phosphorus pentasulfide (P₂S₅) and an alcohol or phenol to form an O,O-dihydrocarbyl phosphorodithioic acid. The alcohol which reacts to provide the R⁴³ and R⁴⁴ groups may be a mixture of alcohols, for instance, a mixture of isopropanol and 4-methyl-2-pentanol, and in some aspects, a mixture of a secondary alcohol and a primary alcohol, such as isopropanol and 2-ethylhexanol. The resulting acid may be reacted with a basic metal compound to form the salt. The metal M, having a valence n, generally is aluminum, lead, tin, manganese, cobalt, nickel, zinc, or copper, and in many cases, zinc, to form zinc dialkyldithiophosphates (ZDP). Such materials are well-known and readily available to those skilled in the art of lubricant formulation. Suitable variations to provide good phosphorus retention in an engine are disclosed, for instance, in U.S. Pat. No. 7,772,171 B2.

Examples of materials that may serve as anti-wear agents include phosphorus-containing antiwear/extreme pressure agents such as metal thiophosphates as described above, phosphoric acid esters and salts thereof, phosphorus-containing carboxylic acids, esters, ethers, and amides; and phosphites. In certain embodiments, a phosphorus antiwear agent may be present in an amount to deliver from about 0.01 to about 0.2, or from about 0.015 to about 0.15, or from about 0.02 to about 0.1, or from about 0.025 to about 0.08 percent phosphorus. The antiwear agent may be a zinc dialkyldithiophosphate (ZDP). For ZDP, which may contain 11 percent P (calculated on an oil free basis), suitable amounts to incorporate into the composition may include from about 0.09 to about 0.82 weight percent. Non-phosphorus-containing anti-wear agents include borate esters (including borated epoxides), dithiocarbamate compounds, molybdenum-containing compounds, and sulfurized olefins.

Other materials that may be used as anti-wear agents include tartrate esters, tartramides, and tartrimides. Examples include oleyl tartrimide (the imide formed from oleylamine and tartaric acid) and oleyl diesters (from, e.g., mixed C₁₂-C₁₆ alcohols). Other related materials that may be useful include esters, amides, and imides of other hydroxy-carboxylic acids in general, including hydroxy-polycarboxylic acids, for instance, acids such as tartaric acid, citric acid, lactic acid, glycolic acid, hydroxy-propionic acid, hydroxyglutaric acid, and mixtures thereof. These materials may also impart additional functionality to a lubricant beyond antiwear performance. These materials are described in greater detail in U.S. Pat. No. 7,651,987 B2 and PCT Publication WO 2010/077630 A1. Such derivatives of (or compounds derived from) a hydroxy-carboxylic acid, if present, may typically be present in the lubricating composition in an amount of from about 0.1 weight % to about 5 wt. %, or from about 0.2 wt. % to about 3 wt. %, based on the total weight of the composition.

### Ashless Antioxidant

In certain embodiments, lubricating compositions described herein may comprise an ashless antioxidant in an amount of from 0.1 wt. % to 5 wt. %, based on the total weight of the composition.

Antioxidants encompass phenolic antioxidants, which may be hindered phenolic antioxidants, one or both ortho positions on a phenolic ring being occupied by bulky groups such as t-butyl. The para position may also be occupied by a hydrocarbyl group or a group bridging two aromatic rings. In certain aspects, the para position is occupied by an ester-containing group, such as, for example, an antioxidant of the formula (XI):

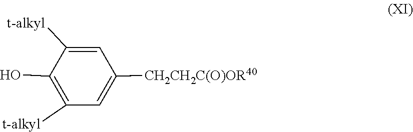

wherein R⁴⁰ is a hydrocarbyl group such as an alkyl group containing, e.g., 1 to 18, or 2 to 12, or 2 to 8, or 2 to 6 carbon atoms; and t-alkyl can be a t-butyl moiety. Such antioxidants are described in greater detail in U.S. Pat. No. 6,559,105 B2.

Antioxidants also include aromatic amines. In one aspect, an aromatic amine antioxidant can comprise an alkylated diphenylamine such as nonylated diphenylamine or a mixture of a di-nonylated and a mono-nonylated diphenylamine, or an alkylated phenylnaphthylamine, or mixtures thereof.

Antioxidants also include aromatic amines. In one aspect, an aromatic amine antioxidant can comprise an alkylated diphenylamine such as nonylated diphenylamine or a mixture of a di-nonylated and a mono-nonylated diphenylamine, or an alkylated phenylnaphthylamine, or mixtures thereof.

Antioxidants also include sulfurized olefins such as monosulfides or disulfides or mixtures thereof. These materials generally have sulfide linkages of 1 to 10 sulfur atoms, e.g., 1 to 4, or 1 or 2. Materials which can be sulfurized to form the sulfurized organic compositions of the present technology include oils, fatty acids and esters, olefins and polyolefins made thereof, terpenes, or Diels-Alder adducts. Details of methods of preparing some such sulfurized materials can be found in U.S. Pat. Nos. 3,471,404 and 4,191,659.

Molybdenum compounds can also serve as antioxidants, and these materials can also serve in various other functions, such as antiwear agents or friction modifiers. U.S. Pat. No. 4,285,822 discloses lubricating oil compositions containing a molybdenum- and sulfur-containing composition prepared by combining a polar solvent, an acidic molybdenum compound and an oil-soluble basic nitrogen compound to form a molybdenum-containing complex and contacting the complex with carbon disulfide to form the molybdenum- and sulfur-containing composition.

Other materials that may serve as antioxidants include titanium compounds. U.S. Pat. No. 7,727,943 B2 discloses a variety of titanium compounds, including titanium alkoxides and titanated dispersants, which materials may also impart improvements in deposit control and filterability. Other titanium compounds include titanium carboxylates such as neodecanoate.

Typical amounts of antioxidants will, of course, depend on the specific antioxidant and its individual effectiveness, but illustrative amounts of each individual antioxidant or the total of all antioxidants can range from about 0.01 to about 5 wt. %, from about 0.15 to about 4.5 wt. %, from about 0.2 to about 4 wt. %, or from 0.8 to about 2.8 wt. %, based on the weight of the total composition.

### Viscosity Modifiers

In certain embodiments, lubricating compositions described herein may comprise a polymeric viscosity modifier in an amount of from 0.1 wt. % to 3 wt. %, based on the total weight of the composition.

Viscosity modifiers (VM) and dispersant viscosity modifiers (DVM) are well known. Examples of VMs and DVMs may include polymethacrylates, polyacrylates, polyolefins, hydrogenated vinyl aromatic-diene copolymers (e.g., styrene-butadiene, styrene-isoprene), styrene-maleic ester copolymers, and similar polymeric substances including homopolymers, copolymers, and graft copolymers. The DVM may comprise a nitrogen-containing methacrylate polymer, for example, a nitrogen-containing methacrylate polymer derived from methyl methacrylate and dimethylaminopropyl amine.

Examples of commercially available VMs, DVMs and their chemical types may include the following: polyisobutylenes (such as Indopol™ from BP Amoco or Parapol™ from ExxonMobil); olefin copolymers (such as Lubrizol™ 7060, 7065, and 7067 from Lubrizol and Lucant™ HC-2000L and HC-600 from Mitsui); hydrogenated styrene-diene copolymers (such as Shellvis™ 40 and 50, from Shell and LZ® 7308, and 7318 from Lubrizol); styrene/maleate copolymers, which are dispersant copolymers (such as LZ® 3702 and 3715 from Lubrizol); polymethacrylates, some of which have dispersant properties (such as those in the Viscoplex™ series from RohMax, the Hitec™ series of viscosity index improvers from Afton, and LZ® 7702, LZ® 7727, LZ® 7725 and LZ® 7720C from Lubrizol); olefin-graft-polymethacrylate polymers (such as Viscoplex™ 2-500 and 2-600 from RohMax); and hydrogenated polyisoprene star polymers (such as Shellvis™ 200 and 260, from Shell). Viscosity modifiers that may be used are described in U.S. Pat. Nos. 5,157,088, 5,256,752 and 5,395,539. The VMs and/or DVMs may be used in the functional fluid at a concentration of up to 20 wt. %. Concentrations of 1 to 12 wt. %, or 3 to 10 wt. %, based on the weight of the total lubricant composition may be employed.

Other performance additives such as corrosion inhibitors, metal deactivators, friction modifiers, foam inhibitors, and pourpoint depressants may be present in the compositions described herein.

Corrosion inhibitors include trialkyl borate esters, polyhydric alcohols (as described in WO 2006/047486 A1), octyl octanamide, condensation products of dodecenyl succinic acid or anhydride and a fatty acid such as oleic acid with a polyamine. In one embodiment, the corrosion inhibitors include the Synalox® (a registered trademark of The Dow Chemical Company) corrosion inhibitor. The Synalox® corrosion inhibitor may be a homopolymer or copolymer of propylene oxide. The Synalox® corrosion inhibitor is described in more detail in a product brochure with Form No. 118-01453-0702 AMS, published by The Dow Chemical Company. The product brochure is entitled “SYNALOX Lubricants, High-Performance Polyglycols for Demanding Applications.”

The lubricating composition may further include metal deactivators, including derivatives of benzotriazoles (such as tolyltriazole), dimercaptothiadiazole derivatives, 1,2,4-triazoles, benzimidazoles, 2-alkyldithiobenz-imidazoles, or 2-alkyldithiobenzothiazoles; foam inhibitors, including copolymers of ethyl acrylate and 2-ethylhexylacrylate and copolymers of ethyl acrylate and 2-ethylhexylacrylate and vinyl acetate; demulsifiers including trialkyl phosphates, polyethylene glycols, polyethylene oxides, polypropylene oxides and (ethylene oxide-propylene oxide) polymers; and pour point depressants, including esters of maleic anhydride-styrene, polymethacrylates, polyacrylates or polyacrylamides.

The lubricating composition may further include friction modifiers, including esters, amides and imides of fatty acids, such as glycerol mono-oleate, vegetable oil trigylcerides, oleyl amide, oleyl tartrimide, malimide, oleyl glycolate; alkoxylated fatty amies, such as ethoxylated tallow amine; and molybdenum compounds. Friction modifiers may be present in the lubricating composition in an amount 0.1 to 1.5 weight % of the composition, or 0.15 to 0.8 weight %, or 0.3 to 0.65 weight %.

In different embodiments, the lubricating composition may be suitable for a gasoline or diesel engine. Such embodiments may have a composition as described in the following table, and the ranges presented in the table may be interchangeable between the embodiments:

| | Embodiments (weight %) | | | |
|---|---|---|---|---|
| Additive | A | B | C | D |
| Salicylate Detergent | 0.1 to 5 | 0.3 to 4.5 | 0.5 to 2.5 | 0.8 to 1.8 |
| Other Calcium | 0 to 2 | 0 to 1.4 | 0.1 to 1.2 | 0 to 0.9 |
| Overbased Detergent | | | | |
| Overbased Magnesium- | 0 to 2.8 | 0.15 to 1.6 | 0.2 to 0.9 | 0.25 to 0.65 |
| based detergent | | | | |
| PIBsuccinimide | 0 to 10 | 0.5 to 6 | 1.1 to 4.5 | 1.5 to 6 |
| Dispersant | | | | |
| Borated Dispersant | 0 to 10 | 0 to 5 | 0.5 to 3 | 0.5 to 1.5 |
| Antiwear agent | 0 to 5 | 0.1 to 2.3 | 0.3 to 1.3 | 0.4 to 1.0 |
| Ashless Antioxidants | 0 to 5 | 0.5 to 4.2 | 0.8 to 2.7 | 1.2 to 2.2 |
| Ashless friction modifier | 0 to 0.5 | 0.1 to 1.6 | 0.3 to 1.1 | 0.3 to 0.8 |
| Polymeric Viscosity | 0 to 10 | 0.2 to 5 | 0.3 to 2 | 0.5 to 1.5 |
| Modifier | | | | |
| Any Other Performance | 0 to 2.5 | 0.01 to 1.7 | 0.15 to 1.2 | 0.2 to 0.85 |
| Additive | | | | |

| | |
|---|---|
| Oil of Lubricating | Balance to 100% |
| Viscosity | |

In different embodiments, the lubricating composition may be suitable for use as a marine diesel cylinder lubricant (MDCL) or a trunk piston engine oil (TPEO). Such embodiments may have a composition as described in the following table, and the ranges presented in the table may be interchangeable between the embodiments:

Embodiments (weight %)

| Additive | A | B | D |
|---|---|---|---|
| Salicylate Detergent | 0 to 20 | 2 to 15 | 5 to 10 |
| Other Calcium | 0 to 20 | 0 to 15 | 0 to 10 |
| Overbased Detergent | | | |
| PIB succinimide | 0.1 to 2 | 0.2 to 1 | 0.25 to 0.5 |
| Dispersant | | | |
| Antiwear agent | 0 to 5 | 0 to 3 | 0 to 2 |
| Ashless Antioxidants | 0 to 5 | 0.1 to 3 | 0.2 to 2 |
| Any Other Performance | 0 to 2.5 | 0.1 to 1.5 | 0.2 to 1 |
| Additive | | | |

| | |
|---|---|
| Oil of Lubricating | Balance to 100% |
| Viscosity | |

In one embodiment, the lubricating composition may have (i) a sulfur content of 0.3 wt. % or less, (ii) a phosphorus content of 0.15 wt. % or less, and (iii) a sulfated ash content of 0.5 wt. % to 1.5 wt. % or less. In one embodiment the lubricating composition may have (i) a sulfur content of 0.3 wt. % or less, (ii) a phosphorus content of 0.09 wt. % or less, and (iii) a sulfated ash content of 0.5 wt. % to 0.9 wt. % or less. In one embodiment, the lubricating composition may have at least one of (i) a sulfur content of 0.2 wt. % to 0.4 wt. % or less, (ii) a phosphorus content of 0.05 wt. % to 0.15 wt. %, and (iii) a sulfated ash content of 0.5 wt. % to 1.5 wt. % or less.

The lubricating compositions disclosed herein may have a kinematic viscosity at 100° C. of from 5 to 12 cSt (mm²/s) and a kinematic viscosity at 40° C. of from 40 to 50 cSt (mm²/s). In another embodiment, the lubricating composition has a kinematic viscosity at 100° C. of from 6 to 10 cSt (mm²/s) and a kinematic viscosity at 40° C. of from 40 to 47 cSt (mm²/s).

Lubricating compositions as described herein may have a high temperature, high shear viscosity (HTHS) of less than 3.0 mPa-s measured at 150° C. per ASTM D4683. In one embodiment, the HTHS viscosity is less than 2.8 mPa-s, such as less than 2.5 mPa-s, from 1.5 to 2.8 mPa-s, from 1.8 to 2.5 mPa-s, or from 1.9 to 2.3 mPa-s.

The lubricating composition including the dispersant additive package may have a TBN of from 4 to 14 mg KOH/g, such as from 5 to 10 mg KOH/g, or 6 to 8 mg KOH/g.

### INDUSTRIAL APPLICATION

The instant disclosure further provides for methods of lubricating an internal combustion engine by supplying the engine a lubricating composition as disclosed herein. In one embodiment, the internal combustion engine is a gasoline-fueled engine. In another embodiment, the internal combustion engine is a diesel engine. Generally, the lubricant is added to the lubricating system of the internal combustion engine, which then delivers the lubricating composition to the critical parts of the engine, during its operation, that require lubrication.

The lubricating compositions described above may be utilized in an internal combustion engine having a surface of steel or aluminum and may also be coated, for example, with a diamondlike carbon (DLC) coating.

The internal combustion engine may be fitted with an emission control system or a turbocharger. Examples of the emission control system include diesel particulate filters (DPF), gasoline particulate filters (GPF), systems employing selective catalytic reduction (SCR), and combinations thereof.

The internal combustion engines may be port fuel injected (PFI) or direct injected. In one embodiment, the internal combustion engine is a gasoline direct injection engine (GDI). Direct injection engines are characterized by injection of the fuel, e.g., gasoline, directly into the cylinder. This is distinct from port fuel injection (PFI) and can result in higher efficiency, higher compression, and/or higher brake mean effective pressure than analogous PFI engines.

In one embodiment, the internal combustion engine is equipped with a turbocharger, a supercharger, or combinations thereof. Turbochargers and superchargers both work to increase the volumetric efficiency of engines, i.e. the volume of air that fills a cylinder relative to the volume of the cylinder. Turbochargers and superchargers work by forcing more air into the cylinder, resulting in higher torque for a given displacement, and hence higher BMEP. In addition to improving the efficiency of an engine, turbochargers and superchargers can increase the likelihood of stochastic pre-ignition, especially at lower speeds.

The lubricating compositions as disclosed herein may be used to lubricate an internal combustion engine operating with a brake mean effective pressure (BMEP) of greater than 12 bars and at a speed of less than 3,000 rpm by supplying to said engine the lubricating composition. In some embodiments, the internal combustion engine is a turbo-charged direct-injection (TDi) engine.

### EXAMPLES

The subject matter disclosed herein may be better understood with reference to the following examples, which are set forth merely to further illustrate the subject matter disclose herein. The illustrative examples should not be construed as limiting the subject matter in any manner.

Example X: Alkyl phenol (a mixture of C_{14,16,18} saturated alkyl groups) was mixed with 100 SUS viscosity mineral oil at a ratio of 70/30 parts by weight. To every 1 mol equivalent of this alkyl phenol was added 1.02 equivalents of a potassium hydroxide aqueous solution at a rate necessary to maintain the batch temperature at 90° C. maximum. After adding the base solution, the mixture was heated to 150° C. and water was removed by distillation. The temperature was adjusted to 125° C. and 1.6 equivalents of carbon dioxide was added to the dehydrated mixture evenly for 12 hours. The resulting mixture of carboxylated alkyl phenate (containing potassium alkyl phenate, potassium alkyl salicylate and mineral oil) had a TBN of 95 mg KOH/g.

Example Y: Example Y was prepared the same as Example X, but with 1.02 equivalents of a sodium hydroxide aqueous solution used instead of the potassium hydroxide aqueous solution. The final mixture had a TBN of 99 mg KOH/g.

Example A1: The carboxylated alkyl phenate reaction mixture of Example X (500 g) was mixed with a small amount of water (2 wt. %) and then dropwise treated with an aqueous solution of sulfuric acid (50 wt. %) at 70° C., over 3 hours to give 1.02 mol equivalents of strong acid. After acid addition was completed, the reaction was stirred for an additional 2 hours at 85° C. The mixture was then heated at 150° C. under agitation until all the water was removed. The resultant slurry was then filtered while hot over a cloth to yield a brown oil (408 g, 127 ppm K).

Examples A2-A11 were prepared in a similar fashion as Example A1 with changes made to the type and concentration of the aqueous mineral acid, and the acid-to-metal ratio applied to either Example X (K-ASA) or Example Y (Na-ASA), as specified in Table 1.

TABLE 1

| | Salicylate | Aqueous Mineral | Acid-to- | Residual Na |
|---|---|---|---|---|
| Example | source | Acid (wt. %) | metal (eq/eq) | or K (ppm) |
| A1 | K-ASA | H₂SO₄ (50%) | 1.02 | K = 127 |
| A2 | Na-ASA | H₂SO₄ (98%) | 1.1 | Na = 11,000 |
| A3 | Na-ASA | H₂SO₄ (50%) | 1.1 | Na = 11 |
| A4 | Na-ASA | H₂SO₄ (50%) | 1.03 | Na = 58 |
| A5 | Na-ASA | H₂SO₄ (85%) | 1.1 | Na = 51 |
| A6 | K-ASA | H₂SO₄ (50%) | 1.1 | K = 100 |
| A7 | K-ASA | H₂SO₄ (50%) | 1.07 | K = 120 |
| A8 | K-ASA | H₂SO₄ (50%) | 1.04 | K = 177 |
| A9 | K-ASA | H₂SO₄ (50%) | 1.03 | K = 78 |
| A10 | K-ASA | H₂SO₄ (50%) | 1.005 | K = 316 |
| A11 | K-ASA | H₂SO₄ (50%) | 1.27 | K = 32 |

Example B1: The carboxylated alkyl phenate reaction mixture of Example X (841 g) was mixed with a small amount of water (2 wt. %) at 70° C. The mixture was then dropwise treated with an aqueous solution of sulfuric acid (42.4 wt. %) at 70° C. over three hours to add the desired 1.1 equivalents of acid (acidic proton pKa <3). After acid addition was completed, the reaction was further stirred for one hour. An excess of water (730 g) was then charged into the flask with stirring to solubilize the salt and the flask was heated to 85° C. The amount of water added to the flask was calculated by determining the solubility limit at 70° C. of the salt product plus an additional 20 wt. %. The stirring in the flask was slowed to 60 rpm for 1 hour and then the stirring was stopped to allow the reaction to separate for 1 hr. The aqueous layer was removed. The organic phase was then collected (727 g) and analyzed for residual K (180 ppm).

Example B2 to B13 were prepared in a similar fashion as Example B1 with changes made to the type and concentration of the aqueous mineral acid, and the acid-to-metal ratio applied to either Example X (ASA Salt=K) or Example Y (ASA Salt=Na), as specified in Table 2.

TABLE 2

| | | | Acid | Acid-to- | | |
|---|---|---|---|---|---|---|
| | ASA | | Conc. | metal | | Na or K |
| Example | Salt | Acid | (wt. %) | (eq/eq) | Separation | (ppm) |
| B1 | K | H₂SO₄ | 42.4 | 1.1 | Fast | K = 180 |
| B2 | Na | H₂SO₄ | 15 | 1.04 | No Separation | N.D. |
| B3 | Na | H₂SO₄ | 50 | 1.04 | No Separation | N.D. |
| B4 | Na | H₃PO₄ | 50 | 1.04 | Slow | N.D. |
| B5 | Na | HCl | 17.1 | 1.04 | No Separation | N.D. |
| B6 | K | H₃PO₄ | 50 | 1.05 | Slow | K = 31 |
| B7 | K | H₃PO₄ | 30 | 1.04 | Slow | K = 80 |
| B8 | K | H₂SO₄ | 30 | 2.1 | Fast | K = 14 |
| B9 | K | H₂SO₄ | 8.6 | 1.04 | No Separation | N.D. |
| B10 | K | H₂SO₄ | 50 | 1.1 | Fast | K = 10 |
| B11 | K | HCl | 15.45 | 1.04 | Fast | K = 26 |
| B12 | K | HCl | 7.3 | 1.1 | Moderate | K = 10 |
| B13 | K | HCl | 15.45 | 1.1 | Fast | K = 13 |

Comparative Example C1: A commercially available calcium alkyl salicylate with 6.1% Ca, 16.4 cSt (100° C.) and a 171 mg KOH/g TBN.

Comparative Example C2: A commercially available calcium alkyl salicylate with 11.5% Ca, 129 cSt (100° C.) and a 330 mg KOH/g TBN.

Example D1: Alkyl salicylic acid from the protonation stage, Example A1, (1218 g) was mixed with 100 viscosity mineral oil (264 g), ethylene glycol (234 g) and hydrated lime (243.6 g). The mixture was heated under nitrogen to 140° C. for 1 hour to remove any water. The mixture was then carbonated at a rate of 380 mL/min for 2 hours. After carbonation was completed, the mixture was heated to 200° C. and stripped under vacuum at 30-40 mmHg. The resulting slurry has 0.8 vol % sediments by centrifuge and was then filtered at high temperature to yield a clear brown product (1522 g) with 7.3% Ca, 32.4 cSt (100° C.) and a 205 mg KOH/g TBN. The product was diluted to 170 mg KOH/g TBN with mineral oil.

Example D2: Alkyl salicylic acid from the protonation stage, Example A1, (1200 g) was mixed with 100 viscosity mineral oil (264 g), ethylene glycol (234 g) and aqueous calcium acetate (86 g, 22 wt. %). Hydrated lime (243.6 g) was charged into the flask and the mixture was heated under nitrogen to 140° C. for 1 hour to remove any water. The mixture was then carbonated at a rate of 380 mL/min for 2 hours. The mixture was then cooled to 120° C. and additional hydrated lime (120 g) was charged into the flask. The mixture was reheated to 140° C. and carbonated again at 380 mL/min for 70 min. The mixture was then cooled again to 120° C. and a third charge of hydrated lime (132 g) was added into the flask. The mixture was reheated to 140° C. and carbonated again at 380 mL/min for 70 min. After carbonation was completed, the mixture was heated to 200° C. and stripped under vacuum at 30-40 mmHg. The resulting slurry has 3.2 vol % sediments by centrifuge and was then filtered at high temperature to yield a clear brown product (1728 g) with 12.5% Ca, 133.7 cSt and a 352 mg KOH/g total TBN. The product was diluted to 320 mg KOH/g TBN with mineral oil.

TABLE 3

| | | Sediment by | | |
|---|---|---|---|---|
| | D2896 TBN | centrifuge | | Salicylic |
| Example | (mg KOH/g) | (vol %) | Filtration | Acid |
| C1 | 170 | Commercially available | | |
| | | overbased salicylate | | |
| C2 | 320 | Commercially available | | |
| | | overbased salicylate | | |
| D1 | 170 | 0.8 | Very Rapid | Example A1 |
| D2 | 320 | 3.2 | Rapid | Example A1 |
| D3 | 203.0 | 0.5 | Very Rapid | Example A5 |
| D4 | 196.1 | 2 | Very Rapid | Example B1 |
| D5 | 205.5 | 0.4 | Very Rapid | Example A1* |
| D6 | 351 | 2.8 | Rapid | Example A8 |
| D8 | 353.2 | 1 | Very Rapid | Example B1 |

*Repeat of A1 on 60 L scale

Lubricating Compositions and test data: A series of engine lubricants in Group I, II or III base oils of lubricating viscosity were prepared containing the detergent composition of the present invention as well as conventional additives including polyisobutenyl succinimide dispersants, polymeric viscosity modifier, overbased detergents (different from that of the invention), antioxidants (combination of phenolic ester and diarylamine), zinc dialkyldithiophosphate (ZDDP), as well as other conventional performance additives as follows for typical Heavy Duty Diesel (HDD) (Table 4), Passenger Motor Car (Table 5) and Marine Diesel (Table 6). The calcium, magnesium, phosphorus, zinc and TBN of each of the examples are also presented in the tables in part to show that each example has a similar amount of these materials and so provide a proper comparison between the comparative and illustrative examples of the present technology.

TABLE 4

| | EX1 | EX2 | EX3 | EX4 |
|---|---|---|---|---|
| Group III Base Oil | Balance to 100% of Composition | | | |
| Example C1 | 1.1 | | | |
| Example C2 | | | 0.68 | |
| Example D1 | | 1.1 | | |
| Example D2 | | | | 0.68 |
| Boron-Free PIB Succinimide² | 2.5 | 2.5 | 2.5 | 2.5 |
| Borated PIB Succinimide³ | | | | |
| Overbased Calcium | | | | |
| Sulfonate⁴ | | | | |
| Overbased Magnesium | 0.3 | 0.3 | 0.3 | 0.3 |
| Sulfonate⁵ | | | | |
| C₃/C₆ Secondary ZDDP | 0.6 | 0.6 | 0.6 | 0.6 |
| Ashless Antioxidant⁶ | 2.6 | 2.6 | 2.6 | 2.6 |
| Soot Dispersant⁷ | 0.8 | 0.8 | 0.8 | 0.8 |
| OCP DVM | 0.2 | 0.2 | 0.2 | 0.2 |
| Styrene Diene polymer | 0.3 | 0.3 | 0.3 | 0.3 |
| Other Additives⁸ | 0.2 | 0.2 | 0.2 | 0.2 |

TABLE 5

| | EX5 | EX6 | EX7 | EX8 |
|---|---|---|---|---|
| Group III Base Oil | Balance to 100% of Composition | | | |
| Example C1 | | | 1.1 | |
| Example C2 | 0.5 | | | |
| Example D1 | | | | 1.1 |
| Example D2 | | 0.5 | | |
| Boron-Free PIB Succinimide² | 2.0 | 2.0 | | |
| Borated PIB Succinimide³ | 1.6 | 1.6 | 3.4 | 3.4 |
| Overbased Magnesium | 0.3 | 0.3 | 0.3 | 0.3 |
| Sulfonate⁵ | | | | |
| C₃/C₆ Secondary ZDDP | 0.9 | 0.9 | 0.8 | 0.8 |
| Ashless Antioxidant⁶ | 1.5 | 1.5 | 1.3 | 1.3 |
| Other Additives⁸ | 1.0 | 1.0 | 2.0 | 2.0 |

TABLE 6

| | EX9 | EX10 | EX11 | EX12 |
|---|---|---|---|---|
| Base Oil | Group I Base Oil | | Group II Base Oil | |
| | Balance to 100% | | Balance to 100% | |
| | of Composition | | of Composition | |
| Example 1 | 4.8 | | 7.2 | |
| (comparative) | | | | |
| Example D1 | | 4.8 | | 7.2 |
| Boron-Free PIB | | | 0.8 | 0.8 |
| Succinimide² | | | | |
| Borated PIB | 0.7 | 0.7 | | |
| Succinimide³ | | | | |
| Overbased Calcium | 6 | 6 | | |
| Sulfonate⁴ | | | | |
| C₃/C₆ Secondary ZDDP | | | 0.8 | 0.8 |
| Ashless Antioxidant⁶ | 0.8 | 0.8 | | |
| Other Additives⁸ | 0.03 | 0.03 | 0.05 | 0.05 |

¹All treat rates are oil free, unless otherwise indicated
²High TBN PIB succinimide dispersant prepared from 1000Mₙ polyisobutylene
³Boron-containing polyisobutenyl succinimide dispersant
⁴Combination of overbased calcium alkylbenzene sulfonate detergents (TBN of 170 and 500 mg KOH/g)
⁵Overbased magnesium alkylbenzene sulfonate (TBN 700 mg KOH/g)
⁶Combination of sulfurized olefins, alkylated diarylamine compounds and hindered phenol ester compounds
⁷Ethylene propylene copolymers functionalized with a mixture of aromatic amines and aromatic polyamines
⁸Other additives include pourpoint depressant, corrosion inhibitor, and anti foam agent

Examples 1 to 12 (shown in Tables 4 to 6) are evaluated in bench and engine tests designed to assess the ability of the lubricant to prevent or reduce deposit formation, provide cleanliness, improve oxidation stability and reduce or prevent acid-mediated wear or degradation of the lubricant. The lubricant samples are subjected to industry standard deposit and oxidation tests including Komatsu Hot Tube (KHT), Pressure Differential Scanning calorimetry (PDSC) (e.g. L-85-99, D6186), and the TEOST 33C (ASTM D6335) and MHT TEOST (ASTM D7097 B) deposit tests, as well as standard evaluation on high frequency reciprocating rigs to asses friction and lubricity. Elementals and test data are summarized below (Tables 7 to 9).

The Komatsu Hot Tube test (KHT) measures the deposit formation tendency of the lubricating composition at high temperature conditions. In the KHT test, high rating means better deposit control performance. The KHT test employs heated glass tubes through which a sample lubricating composition is pumped (5 mL total sample), at 0.31 mL/hour for 16 hours, with an air flow of 10 mL/minute. The glass tube is rated at the end of test for deposits on a scale of 0 (very heavy varnish) to 10 (no varnish).

The HDD and PCMO lubricant compositions were assessed for their frictional and wear performance using a high frequency reciprocating rig (HFRR) equipped with a standard steel ball on steel disk. The following test conditions were utilized: 200 N force, frequency of 20 Hz, 75 minutes duration, and temperature was held at 40° C. for 15 minutes and then ramped at 2° C. per minute to a final temperature of 160° C. (60 minute ramp). Coefficient of friction (COF) is measured virtually continuously during the entire test. The average coefficient of friction is determined by averaging all of the measurements during the temperature ramp phase of the procedure. The test procedure has two phases, an initial isothermal stage followed by a ramp phase; the measured value is the average coefficient of friction during the temperature ramp phase only. The coefficient of friction is the frictional force measured parallel to the reciprocation divided by the applied force.

TABLE 7

| | EX1 | EX2 |
|---|---|---|
| TBN (mg KOH/g) | 9.62 | 9.9 |
| TBN (ASTM D4739) (mg KOH/g) | 6.60 | 6.60 |
| Kinematic Viscosity @ 100° C. (D445) | 7.36 | 7.232 |
| High Temperature High Shear Viscosity | 2.43 | 2.39 |
| (D4683) (cP) | | |
| Calcium (ppm) | 1190 | 1228 |
| Magnesium (ppm) | 490 | 430 |
| Phosphorus (ppm) | 630 | 598 |
| Zinc (PPM) | 700 | 688 |
| TEOST 33C (D6335), Total Deposit(mg) | 34.7 | 36.9 |
| Oxidation Induction Time (min) | 107.8 | 111.2 |
| (L-85-99, OIT) | | |
| KHT (rating 0-10) @ 280° C. | 7 | 8 |
| HFRR Coefficient of friction @ 160° C. | 0.173 | 0.175 |

TABLE 8

| | EX5 | EX6 |
|---|---|---|
| TBN (mg KOH/g) | 7 | 7.1 |
| TBN (ASTM D4739) (mg KOH/g) | 5.9 | 6 |
| Calcium (ppm) | 1075 | 1065 |
| Magnesium (ppm) | 466 | 462 |
| Phosphorus (ppm) | 739 | 745 |
| Zinc (PPM) | 843 | 843 |
| TEOST 33C (D7097 B), Total Deposit(mg) | 25.3 | 26.7 |

TABLE 9

| | EX9 | EX10 | EX11 | EX12 |
|---|---|---|---|---|
| TBN (D2896) (mg KOH/g) | 40.9 | 40.8 | 39.8 | 40.2 |
| Calcium (ppm) | 14353 | 14185 | 14592 | 14419 |
| Phosphorus (ppm) | | | 542 | 590 |
| Zinc (PPM) | | | 624 | 669 |
| Oxidation Induction Time (min) | 171.4 | >300 | 73.9 | 87.8 |
| (D6186, OIT) | | | | |
| KHT (rating 0-10) @ 310° C. | 0 | 7.5 | 2 | 1 |

The data indicates that the lubricant compositions containing the detergent additive of the present subject matter provides equivalent or better cleanliness, oxidation control and/or frictional properties as compared with commercially available overbased alkylsalicylate detergents.

Except in the Examples, or where otherwise explicitly indicated or required by context, all numerical quantities in this description specifying amounts of materials, reaction conditions, molecular weights, number of carbon atoms, and the like, are to be understood as modified by the word “about”. It is to be understood that the upper and lower amount, range, and ratio limits set forth herein may be independently combined, and that any amount within a disclosed range is contemplated to provide a minimum or maximum of a narrower range in alternative embodiments (with the proviso, of course, that the minimum amount of a range must be lower than the maximum amount of the same range). Similarly, the ranges and amounts for each element of the subject matter disclosed herein may be used together with ranges or amounts for any of the other elements.

While certain representative embodiments and details have been shown for the purpose of illustrating the subject matter disclosed herein, it will be apparent to those skilled in this art that various changes and modifications may be made therein without departing from the scope of the subject matter. In this regard, the scope of the invention is to be limited only by the following claims.

1. A process comprising:
a. contacting an alkyl phenol with an alkali metal base compound and carbon dioxide at a temperature of from 120° C. to 180° C. to form an alkyl salicylic acid salt of the alkali metal base compound;
b. contacting the result of (a) with a mineral acid, in the absence of an additional non-polar hydrocarbyl liquid having a kinematic viscosity of less than 2.5 m²/s at 100° C., wherein the mineral acid and the alkali metal base compound form an alkali metal salt of the mineral acid, and wherein a solvent for the mineral acid is present in an amount sufficient to at least partially solubilize the alkali metal salt of the mineral acid to produce an alkyl salicylic acid and/or a salt thereof; and
c. removing at least 50 wt. % of the solvent and the alkali metal salt of the mineral acid from the result of (b), based on the total amount of solvent and alkali metal salt present in the result of (b).

2. The process of claim 1, wherein said removing the solvent comprises: (i) heating the result of (b) to a temperature sufficient to at least partially vaporize the solvent in the result of (b) and to form a precipitate of the alkali metal salt of the mineral acid, followed by removal of the precipitate, optionally wherein said removal is via filtration; and/or (ii) liquid-liquid phase separation.

3. The process of claim 2, wherein said heating is performed at a temperature of from 60° C. to 160° C., optionally under vacuum.

4. The process of claim 1, wherein the alkali metal base compound is present in an amount of from 0.5 to 1.5 reactive equivalents based on the amount of the alkyl phenol.

5. The process of claim 1, wherein the mineral acid is present in an amount of at least 0.9 equivalents of strong acid, based on the amount of alkali metal base compound.

6. The process of claim 1, wherein a diluent oil is present in step (a) in an amount of from 10 wt. % to 50 wt. %, based on the total weight of all components present in step (a).

7. The process of claim 1, wherein the mineral acid is a polyprotic mineral acid.

8. The process of claim 1, wherein the mineral acid comprises at least one of sulfuric acid, hydrochloric acid, or phosphoric acid.

9. (canceled)

10. The process of claim 1, wherein the alkyl phenol comprises at least one of 4-substituted alkyl phenol, 2-substituted alkyl phenol, or 2,4-disubstituted alkyl phenol.

11. The process of claim 1, wherein the alkali metal base comprises at least one of sodium hydroxide, potassium hydroxide, or lithium hydroxide.

12. The process of claim 1, further comprising: (d) contacting the result of (c) with: (i) an alkaline earth metal oxide/hydroxide and/or a group II metal oxide/hydroxide, or a reactive equivalent thereof; (ii) carbon dioxide; and (iii) an alcohol, polyol, and/or (poly)ether; to produce an overbased detergent having a total base number of from 130 mg KOH/g to 600 mg KOH/g on an active basis.

13. The process of claim 12, wherein the alkaline earth metal oxide/hydroxide and/or group II metal oxide/hydroxide, or a reactive equivalent thereof, is added in an amount of 1.1 to 8 reactive equivalents based on the alkyl salicylic acid.

14. The process of claim 12, wherein the alkaline earth metal oxide/hydroxide comprises at least one of calcium oxide, calcium hydroxide, magnesium oxide, or magnesium hydroxide.

15. The process of claim 12, wherein the group II metal oxide/hydroxide comprises zinc oxide.

16. The process of claim 12, wherein the alcohol, polyol, and/or (poly)ether is present in an amount of from 5 wt. % to 30 wt. %, based on the total weight of all components present in step (d).

17. The process of claim 12, wherein the alcohol, polyol, and/or (poly)ether comprises at least one of ethylene glycol or propylene glycol.

18. (canceled)

19. The process of claim 12, wherein the overbased detergent has a metal ratio of from 1.5 to 10.

20. An alkyl salicylic acid prepared according to the process of claim 1.

21. (canceled)

22. An overbased detergent prepared according to the process of claim 12.

23. A lubricating composition comprising the overbased detergent of claim 22.

24. (canceled)

25. (canceled)

26. (canceled)

27. (canceled)

28. (canceled)
